(19) United States
(12) Patent Application Publication
(10) Pub. No.: US 2005/0272059 A1
(43) Pub. Date: Dec. 8, 2005

(54) Method for providing microarrays

(76) Inventors: Xiaolian Gao, Houston, TX (US); Xiaoming Wang, Burr Ridge, IL (US); Jerry Liu, Houston, TX (US)

Correspondence Address: VINSON & ELKINS, L.L.P., 1001 FANNIN STREET, 2300 FIRST CITY TOWER, HOUSTON, TX, 77002-6760, US

(21) Appl. No.: 11/056,782

(22) Filed: Feb. 11, 2005

Related U.S. Application Data
(60) Provisional application No. 60/544,896, filed on Feb. 12, 2004.

(51) Int. Cl. C12Q 1/68; G06F 19/00; G01N 33/48; G01N 33/50

(52) U.S. Cl. 435/6; 702/20

(57) ABSTRACT

An interactive method of providing an array of nucleic acid sequences in which a remote user enters a query to generate a listing of desired sequence probes, which are then selected and returned to the host for use in producing a custom microarray designed by a remote user.

### CROSS-REFERENCE TO RELATED APPLICATIONS

This application claims benefit of U.S. Provisional No. 60/544,896, filed Feb. 12, 2004.

### STATEMENT REGARDING FEDERALLY SPONSORED RESEARCH OR DEVELOPMENT

N/A

### BACKGROUND OF THE INVENTION

Microarrays containing genetic probes are typically marketed in a catalog arrangement in which standard microarrays are available that contain probes that will detect mRNA or DNA from various organisms or viruses and that will detect expression of certain genes or mutations associated with various disease states.

Custom microarrays are also available in which a consumer may need particular probes designed for assays for which no catalog microarray is available. Custom arrays are typically more expensive and take longer to create.

### SUMMARY

The present disclosure addresses certain deficiencies in the art by providing a system in which a user is able to interactively design a custom microarray that is fabricated quickly and inexpensively. A user is able to select gene probes for any organism and to select particular families of genes based on function, location, or other criteria in order to construct a completely custom array. In this way, a user may design multiple arrays for a single study, or may include several smaller studies on a single microarray device as needed.

The present invention may be described, therefore, in certain embodiments as an interactive method of providing an array of nucleic acid sequences. The method may include the steps of preparing a database of gene probes wherein each gene probe is effective to identify a specific target gene from a particular organism, and further wherein each of the gene probes is identified by one or more identifiers; providing an electronic connection to the database effective to allow a user to electronically query the database by the identifiers; providing means for storing a list of gene probes or probe identifiers; transmitting a collection of selected gene probes or probe identifiers from the user to the host; and producing a nucleic acid probe array that comprises the selected gene probes. Gene probe sets are preferably provided for any organism, including but not limited to human, mouse, rat, Xenopus, Drosophila and various bacterial genes, for example. Gene probes for other organisms are added to the database as gene sequences become available.

Gene probes may be selected by a variety of identifiers including, but not limited to accession numbers, accession alias numbers, disease names, disease synonym names, gene functions, gene names, gene symbols, gene descriptions, PFAM names, gene ontology (GO) molecular function terms, or locus I.D. numbers. The gene probes preferably include 1-5 nucleic acid sequences, and may include 1, 2, 3, 4, 5 or more genetic probes directed to the selected target. It is understood that more than five probes may be included in a gene probe set, but that 5 is a preferred number.

The selected list may be stored by any means known in the art, including a storage device on a host server, or a software command that directs storage of the list on the user's remote computer. The user would then choose the storage media which may be a hard disk, CD-ROM, zip drive, floppy disk, or any other storage media known in the art. In preferred embodiments the nucleic acid probes of the microarray are nucleic acid polymers of from 40 to 50 bases in length, or they may be probes of 10-100 bases, including 12, 15, 18, or any integer between 10-100 inclusive, in length if needed for any particular application. In preferred embodiments, however, the probes are designed to hybridize to their respective target genes in a region within 1000-1500 bases of the 3′ end of the cDNA of the target gene where poly adenylated mRNAs are present. For organisms where the mRNAs do not contain a poly-A tail, there is no need to impose a positional bias of probe location. It is understood then, that all the probes in a chosen gene probe set will hybridize to this region of the target nucleic acid. The probes are also preferably optimized for Tm, C+G content, probe secondary structure, and dimerization tendency. It is understood that the microarray may also contain control sequences. Control sequences are known in the art and are included to monitor wash conditions, target labeling, hybridization and possibly to aid in quantitation of target nucleic acids.

In preferred embodiments, the nucleotide probes of the array are made by synthesis of the nucleic acid probes in situ on a solid substrate comprising isolated sites. Most preferably the nucleotide probes are synthesized by steps that include (a) addition of protecting groups to the reactive species within the isolated sites wherein the protecting groups are not photo-labile; (b) contacting the isolated sites with photo-generated agent precursors; (c) irradiating a selected subset of the isolated sites effective to generate active agents from the precursors within the selected subset and to deprotect reactive chemical groups within the selected subset; (d) contacting the isolated sites with a selected nucleic acid monomer comprising a free reactive chemical group and a protected reactive chemical group under conditions in which the free reactive chemical group of the monomer bonds to the deprotected reactive chemical group of the selected subset; and (e) repeating steps b-d until the selected nucleic acid probes are synthesized.

Probes or probe sets may also be synthesized elsewhere, on an automated DNA synthesizer for example, or retrieved from a pre-made library of oligonucleotides and spotted onto the array substrate by methods known in the art. The oligonucleotides may be known sequences or may be custom sequences designed by a user or host. Any such methods of producing the arrays are contemplated by the present disclosure.

The present invention may also be described in certain embodiments as a system for providing custom nucleic acid microarrays. The system preferably includes a host computer with a memory storage media; a database of gene probes stored on the storage media wherein each gene probe is associated with one or more identifiers; a user connection configured to allow remote users to connect to the host computer and to access the database; software means to allow a remote user to query the database to generate a list of gene probes; and means for remotely selecting listed gene probes; wherein the selected gene probes are provided on a custom manufactured microarray. The interface to a remote computer may be an internet web interface, or though any means known in the art, including but not limited to a modem connection which may be through a telephone or cable connection to an internet, for example, and may be a wireless connection in certain embodiments. The system may further include a media for storing a user's selected gene probes until a further search is done, or until the microarray is ready to be ordered. Storage means may include a storage disk on the host computer network or server, or it may be software that directs storage of the gene probe list on the user's own computer.

Throughout this disclosure, unless the context dictates otherwise, the word “comprise” or variations such as “comprises” or “comprising,” is understood to mean “includes, but is not limited to” such that other elements that are not explicitly mentioned may also be included. Further, unless the context dictates otherwise, use of the term “a” may mean a singular object or element, or it may mean a plurality, or one or more of such objects or elements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is an example of a login screen according to the present disclosure.

FIG. 2A-2B is an example of a search page from which a basic search for a human gene may be searched by accession number. In order to execute a search, a user enters the accession number of the gene of interest into the appropriate box and clicks on the “go” button.

FIG. 3A-3B is an example of a basic search according to the present disclosure in which a search has been conducted for human genes by disease name and the disease searched is “diabetes.”

FIG. 4A-4D is an example of a batch search for the species “human” searched by PFAM name.

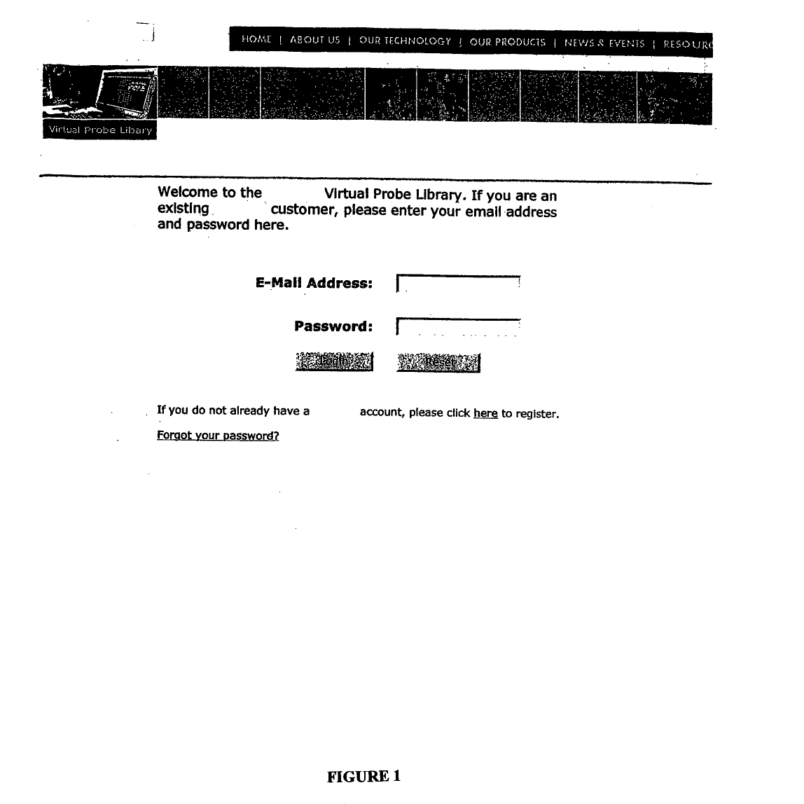

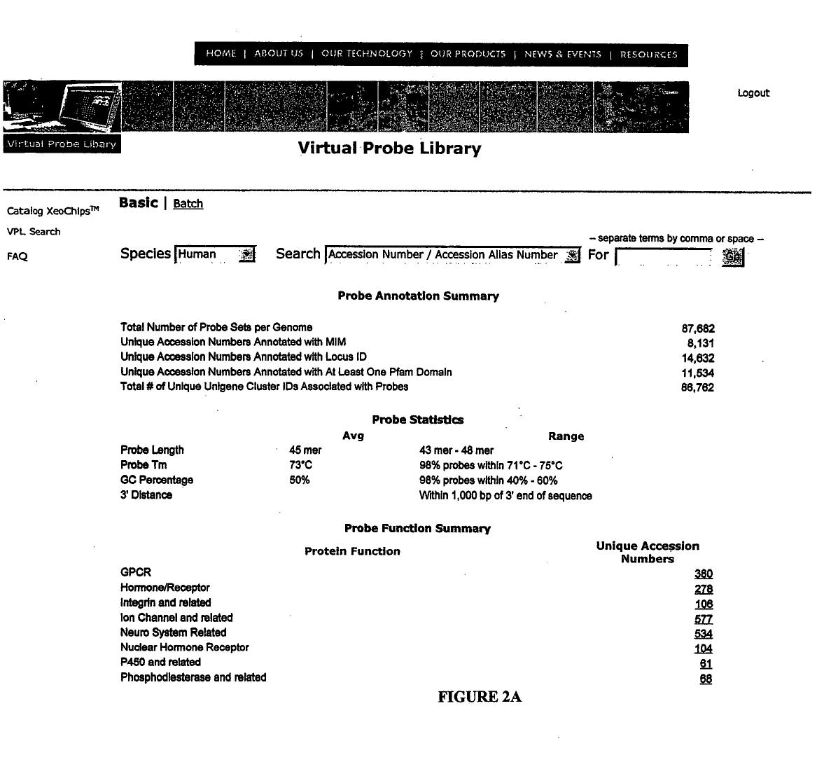

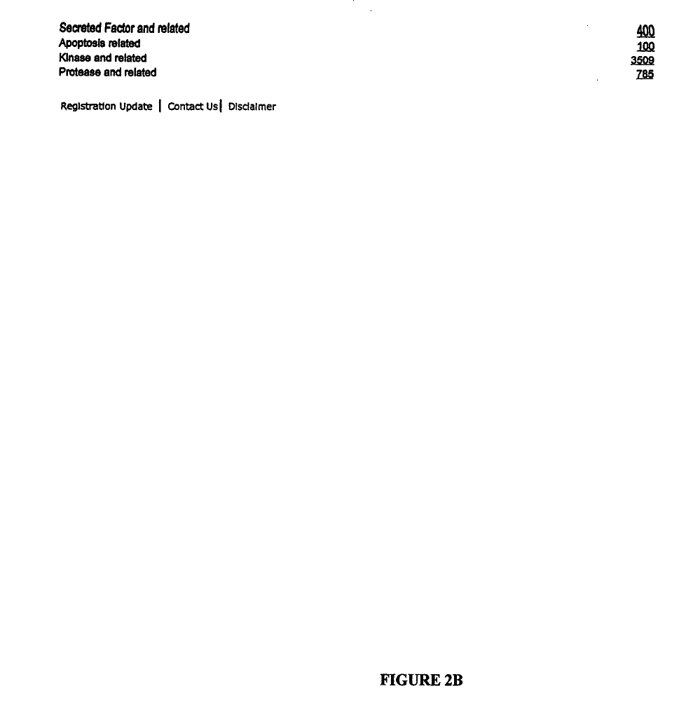

### DETAILED DESCRIPTION

An aspect of the present disclosure is a system and method of electronic marketing of a custom microarray through a system that has been named the virtual probe library (VPL). The VPL is a relational database that manages DNA oligonucleotide expression probes designed for a given genome. It is an aspect of the invention that a provider of the disclosed methods designs sets of probes that are effective for use in microarray technology. The purpose of the VPL is to allow a user to assemble a “probe list” for the genes of interest to the user. The probe list is then submitted to the host practitioner online and is used to synthesize a microarray device, such as a microarray silicon chip or glass slide microarray. By utilizing the VPL, a user only has to specify one or more target genes that are to be the subject of investigation, and then select a pre-designed probe or probe set that will identify the target gene or genes using a microarray device. To help facilitate queries of the VPL, a biological functional search function can be integrated with each probe set.

In preferred embodiments, the database contains probe sets rather than individual probes. A probe set contains a group of probes designed to hybridize to, and thus identify a particular gene. In preferred embodiments, each probe set may have up to five probes (1-5), although the upper limit is a pragmatic choice. Any number of probes may be included in a set, including 10, 20, 30, 40, or even 50 or more, although 5 has been shown to be an efficient number for certain applications. Each individual probe has its own identifier (probe index) while a probe set uses a gene accession number as its identifier (probe accession number). When the database is queried through the VPL, probe sets are identified rather than individual probes. In preferred embodiments, when the user orders a microarray, he or she has an option to specify single or multiple probes per gene.

The probes used in the disclosed method may be of any effective length. For example, probes of from about 10 to about 100 bases, from about 20 to about 100 or even from about 43 to about 48 bases may be provided. The stated ranges are understood to be inclusive of the limits and to include all integers between the upper and lower limits. The term “about” indicates that probes that are slightly longer or slightly shorter than the stated range but that have essentially the same binding characteristics to the target gene would be included in the range. For example, in a probe of 45 bases, a probe of 42, 43, 44, 46, 47, or even 48 bases in length that had essentially the same binding characteristics to the target gene would be included in the term “about 45.” In preferred embodiments the individual probes are about 43-48 nucleotides in length, and preferably about 45 nucleotides (45-mer). The probes are designed for any genome using the Unigene and/or RefSeq databases as the major source for DNA sequences used in probe design (publicly available from the National Center for Biotechnology Information). The probes are preferably designed using the Santa Lucia nearest neighbor thermodynamics algorithm to calculate probe target Tm (Santa Lucia, J., Jr. A Unified View of Polymer, Dumbbell, and Oligonucleotide DNA Nearest-neighbor Thermodynamics. Proc. Natl. Acad. Sci. and normalized Lempel-Ziv complexity are also taken into the consideration (Lempel and Ziv, IEEE Trans. Info. Theory 22: 75-88 (1976)).

It is understood that any genetic sequence databases may be used in the design of the probes, however, these mentioned are convenient and readily available at the time of this disclosure. Other databases known in the art, and those developed subsequent to the filing of the present application may also be used as appropriate.

In the practice of certain preferred embodiments, when a user connects to the host server, he or she may be directed to a login page. The user preferably creates an account by entering a username and password. In this way, the user's administrative files (name, address, billing, shipping information, etc.) as well as the user's confidential probe list or gene list may be stored under the username for future use or reference. After logging into the system, the user is directed to the virtual probe library design page. From this page, the user may query the VPL through a series of choices selected by user input devices such as mouse clicks or keyboard inputs, for example. It is understood that various types of user input devices such as touch screens, voice recognition, and the like, may be used that are not limited to a keyboard or computer mouse, and that any such input device is acceptable in the practice of the disclosed inventions.

The first selection a user may make is to select the organism of interest. Preferably a drop down menu is used to select the organism among those available. For example, a drop down menu labeled “organism” may include a list such as human, mouse, rat, Xenopus, Drosophila, bacterial genome, or any other organism for which gene sequences are available in the probe library database.

After selecting the organism, the user may select the type of query, either “basic,” which is typically a single word query, or a multiple term “batch” query. The basic query page provides a space in which the query is typed or pasted by the user. The batch query page allows the user to submit a list of query terms that must belong to one type of query, and also may allow the importation of a file containing the list of terms by providing a window in which to enter a file name and location for importation into the query space. When importing a file or entering query terms, each query term should end with a new line, and they should be saved as a text file, a spreadsheet file or any other appropriate file format. In certain preferred embodiments, the VPL allows batch searches with a maximum of 50 terms for each batch query, although larger searches are contemplated.

The user also selects the type of search terms to be used. This again is preferably provided by a drop down menu with a list of the types of search terms that can be accepted. An example of such terms includes accession numbers/accession alias numbers, disease name/disease synonym name, gene functions, gene names, gene symbol, gene description, PFAM names, gene ontology (GO) molecular function terms, and locus I.D. numbers. Other types of search terms may also be added by the practitioner and the cited list is merely for purposes of illustration.

Examples of types of queries in preferred embodiments include:

i) Query by accession number/accession alias number. This query is intended for NCBI cDNA sequence accession numbers. The reported results typically give the representative accession number for the gene of interest. “Accession aliases” are alternate accession numbers in a Unigene cluster that refer to the same gene product. UniGene is an experimental system for automatically partitioning GenBank sequences into a non-redundant set of gene-oriented clusters. Each UniGene cluster contains sequences that represent a unique gene, as well as related information such as the tissue types in which the gene has been expressed and chromosomal, physical map location. It is understood that probe set gene annotations may be updated from time-to-time to better match current records at NCBI.

ii) Query by disease name/disease synonym name. Searches may be conducted for genes associated with different genetic disorders according to the Mendelian Inheritance in Man (MIM) classifications. In preferred embodiments, certain accession numbers are associated with a record in the Mendelian Inheritance in Man database. Human probe sets have been developed by the present inventors, including approximately 8,200 human probe sets associated with this database, and each probe set targets a unique transcript sequence. This association allows a user to query probes in VPL using disease names. The official disease name and layman disease name are equally evaluated when querying disease related probes. A user may use the key word “leukemia” or “T cell lymphoma” for example, to locate the genes of interest. These disease names should appear in the MIM database in order to obtain a probe list.

iii) Query by Gene Name/Symbol. A query may be a gene symbol, a gene symbol alias, historically used symbols, or gene names defined by HUGO gene nomenclature committee in order to obtain a probe list. VPL allows the user to use an officially approved gene symbol, symbol alias, used symbol, gene name, or gene description to query the VPL, as long as these terms are in the HUGO gene nomenclature hosted database. It may happen that a certain query term does not belong to any one of these categories. In that case, the user may have to know the gene accession number or other identifier to query the VPL.

iv) Query by Gene Ontology (GO) terms. The GO terms are defined by the Gene Ontology consortium and can be found in the GO database. Current GO term structure at VPL belongs to the GO molecular function tree. For example, when a user uses the query term “death” to search probes, the VPL returns any GO terms that contain a key word “death”. If the term is in an end node of the tree, VPL only shows the probe set number within this node, such as GO:0005037. However, if the term is at a higher hierarchy of the tree, VPL will show both the number of probe sets within this node and child node number of this term. For example, there is one probe set at the node GO:0005035 and there are nine child nodes under this level.

For example

| | GO ACC (# of | | |
|---|---|---|---|
| Query Term | Child) | GO Term | # of Probe Set |
| death | GO:0005035 (+9) | death receptor | 1 |
| death | GO:0005037 | death receptor | 4 |
| | | adaptor protein | |
| death | GO:0005038 | death receptor | 3 |
| | | interacting protein | |
| death | GO:0005039 | death receptor- | 2 |
| | | associated factor | |
| death | GO:0005040 | decoy death | — |
| | | receptor | |
| death | GO:0005123 | death receptor | 1 |
| | | binding | |

v) Query by Pfam name. Pfam names may also be entered. Pfam HMM names are defined in the Pfam database hosted by Washington University. The Pfam HMM names refer to conserved protein families and protein domains. The present inventors currently have approximately 12,400 human probe sets that are associated with Pfam records. This association allows the user to search for probes in the VPL using Pfam domain names. The user is preferably required to know HMM names to do a Pfam query. The HMM names can be found at the Pfam database in the public domain. To facilitate Pfam query efficiency, VPL provides the most commonly used function summary table at the front page of each genome as a shortcut to the desired probes. The probe function summary table was generated by grouping probes that represent similar or related function molecules defined by HMMs at the protein level. For example, the “kinase and related” group contains kinase, kinase inhibitor and activators, while the GPCR group contains all 7 transmembrane receptors.

In preferred embodiments, when searching for accession numbers, the RefSeq mRNA accession numbers (prefix NM) are used to denote the reference sequence (or representative sequence) of a gene cluster and this type of accession number is preferably returned in the query results. It is also understood that not all of the genomes support a search with all the available query types. The user may, therefore, search with one type of query, such as gene function, and may then do subsequent searches using disease name or GO molecular function terms, for example. A user may save the list of probes that have been selected both to his or her own computer, and to the “shopping cart” provided by the host server. In this way, when the user logs in again, the previous search results are available. Therefore, in preferred embodiments, when a search is complete, it is recommended that a user download the list to a spreadsheet file such as CSV or Excel file in the user's own computer (for reference purposes) and place the selected probes into the “shopping cart” file where they are saved under the user's account. The probe list may then be submitted online to place an order for a microarray chip, or the list may be used to determine the number of unique probes available for the gene set of interest prior to ordering.

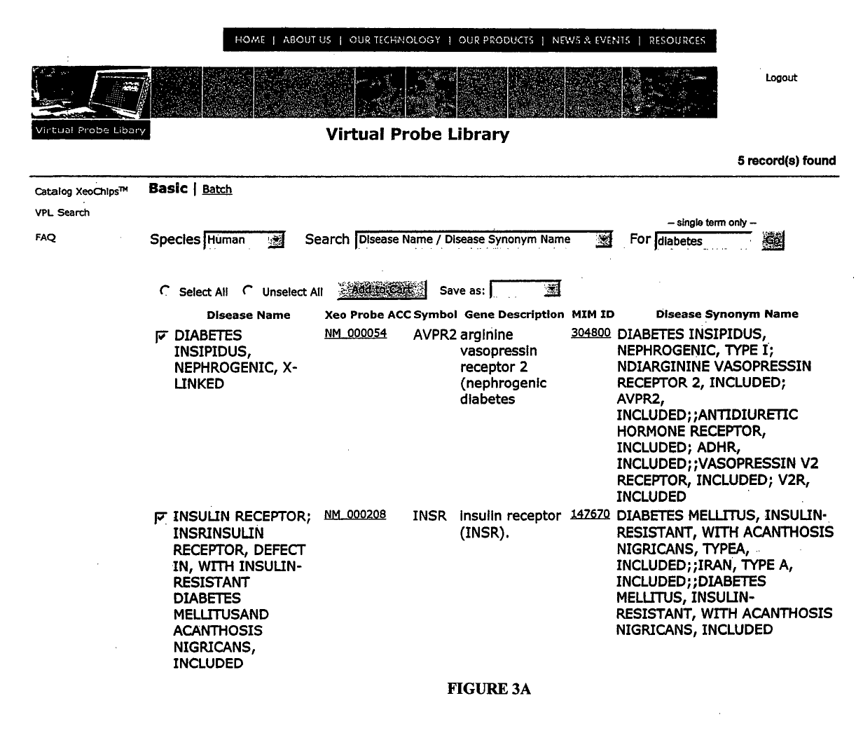

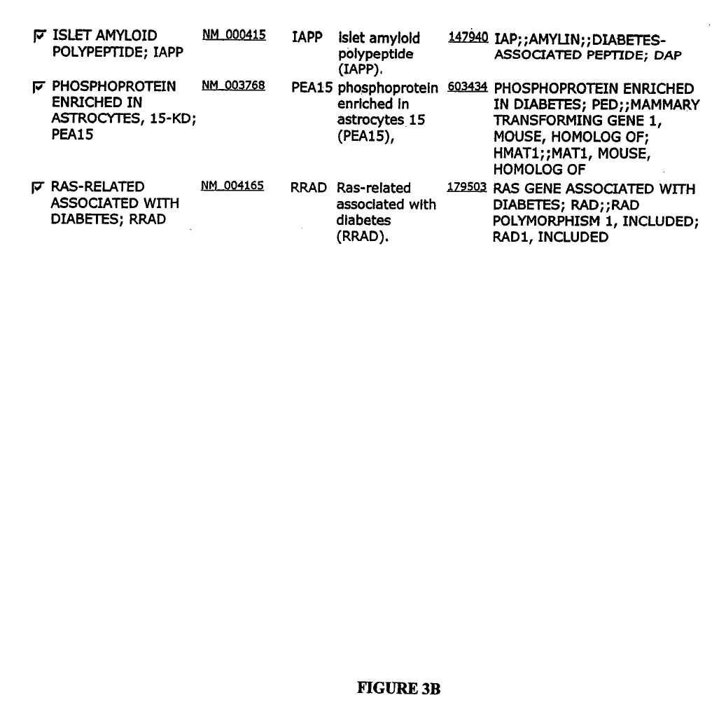

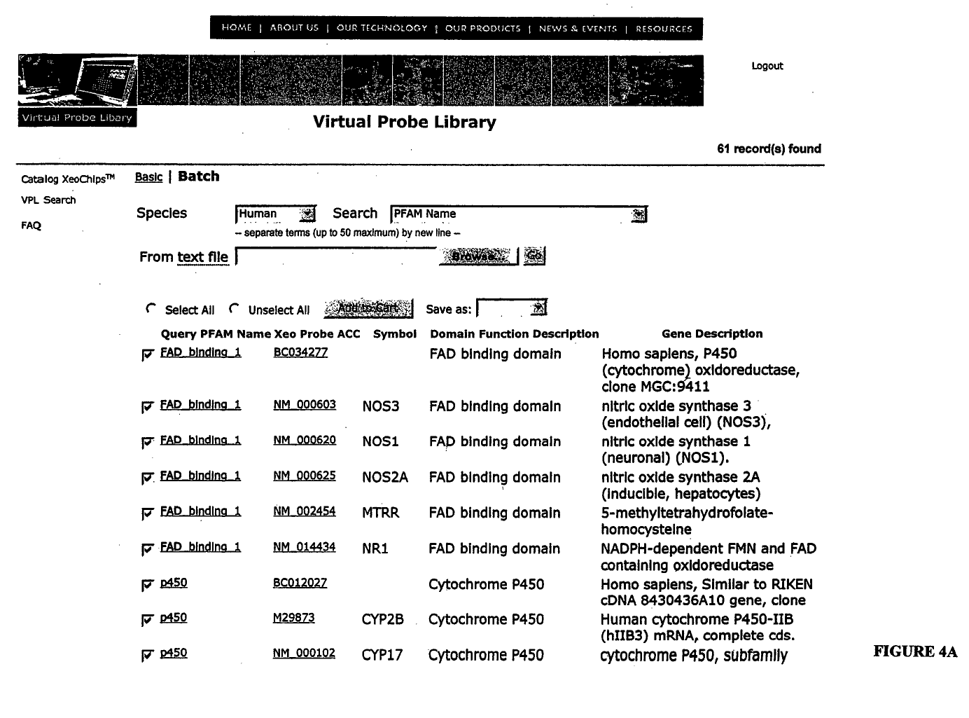

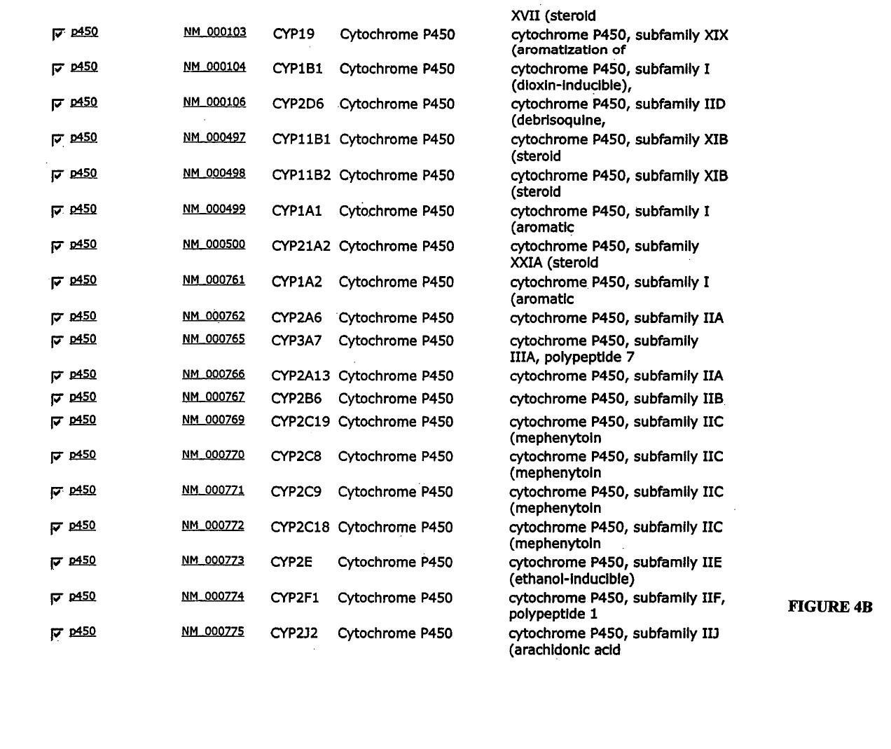

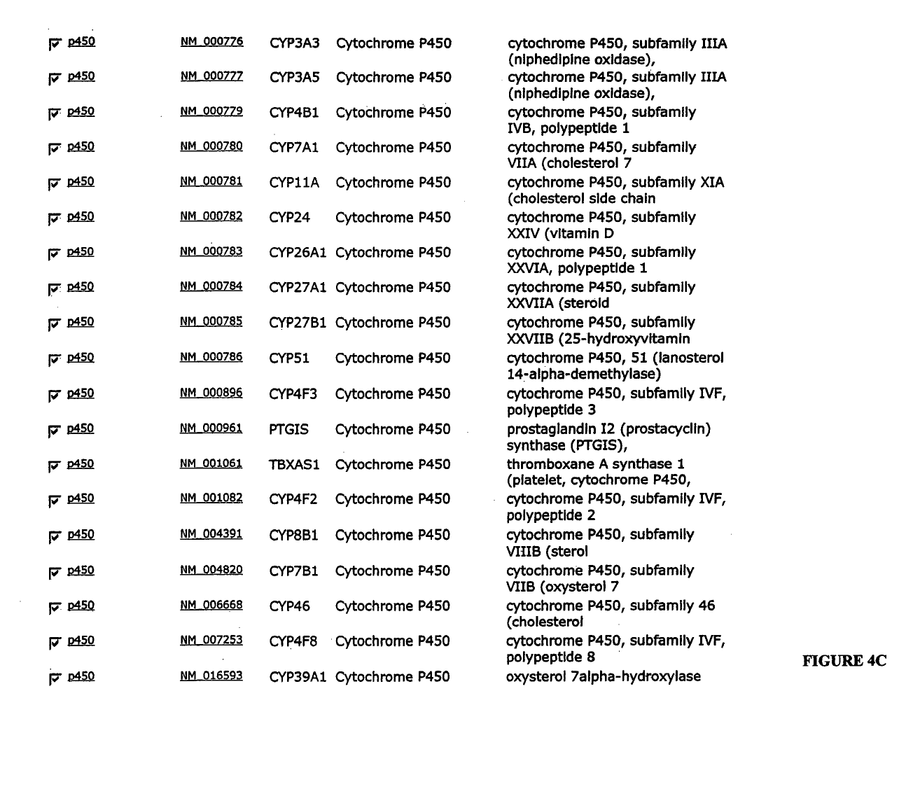

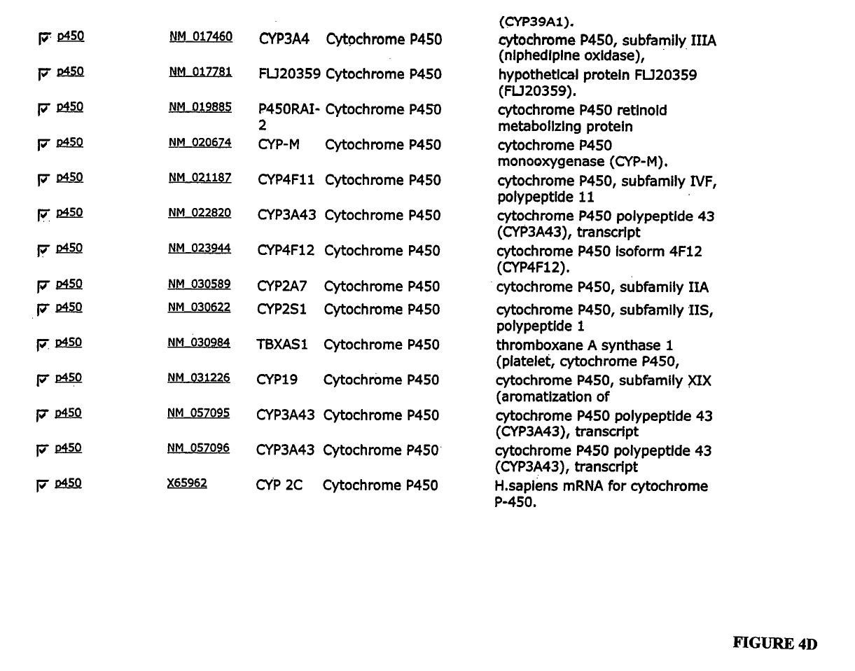

An example of search results is shown in FIG. 3A-B. In this example, a basic query was done for genes in the human genome. The type of query was “disease name/disease synonym name” and the query was “diabetes.” As can be seen in the Figure, the search resulted in 5 probe sets. Each probe set in the results may be selected individually, they may all be selected or they may all be unselected by clicking in the appropriate space or check-box. The selected probes may then be added to the shopping cart or saved on the user's computer under a file name selected by the user. An example of a batch search of human genes by Pfam name is shown in FIG. 4A-D.

In preferred embodiments the microarray is provided on a device such as a silicon chip or glass slide. Preferred chip devices may include any number of elective features per array. For example, a microarray device may include from 100 to 10,000 or more elective features per array. It is understood that the number of features is limited only by the available technology and the type of array desired. A preferred embodiment includes 7947 elective feathers per array device. Selected probes may be replicated as many times as can occupy the remaining space on the chip or slide. Alternatively, probes can be designated for addition across multiple chips if the number of probes in the probe list exceeds the maximum feature number allowed on one array. In this way, a user can specify how probes are to be placed on the array, how many genes may be targeted per array and how many probes per gene are to be used and how many times a probe may be replicated on the array(s). The present inventions thus allow a user to completely custom design his or her own arrays through a digital network system.

In preferred embodiments, the probes are synthesized in solution using in situ generated photo-products as reagents or co-reagents. The use of photogenerated reagents in the synthesis of nucleotide probes in situ on a solid substrate is described in U.S. Pat. No. 6,426,184, incorporated herein in its entirety. As used herein, synthesis in situ is meant to indicate that the probes are synthesized by addition of selected monomers to the substrate and to previously added monomers at the location of the solid substrate where they are to be used, and are not synthesized elsewhere and then attached to the surface after they are synthesized.

These reactions are controlled by irradiation, such as with UV or visible light. Unless otherwise indicated, all reactions occur in solutions of at least one common solvent or a mixture of more than one solvent. The solvent can be any conventional solvent traditionally employed in the chemical reaction, including but not limited to such solvents as CH₂Cl₂, CH₃CN, toluene, hexane, CH₃OH, H₂O, and/or an aqueous solution containing at least one added solute, such as NaCl, MgCl₂, phosphate salts, etc. The solution is contained within defined areas on a solid surface containing an array of reaction sites. Upon applying a solution containing at least one photo-generated reagent (PGR) precursor (compounds that form at least one intermediate or product upon irradiation) on the solid surface, followed by projecting a light pattern through a digital display projector onto the solid surface, photo-generated acid (PGA) forms at illuminated sites; no reaction occurs at dark (i.e., non-illuminated) sites. The PGA modifies reaction conditions and may undergo further reactions in its confined area as desired. Therefore, in the presence of at least one photo-generated reagent (PGR), at least one step of a multi-step reaction at a specific site on the solid surface may be controlled by radiation, such as light, irradiation. At each step of the reaction, (each addition of another monomer) only selected sites in a matrix or array of sites are allowed to react. In preferred embodiments light patterns for effecting the reactions are generated using a computer and a digital optical projector (the optical module). Patterned light is projected onto specific sites on the microarray substrate, where light controlled reactions occur.

It is understood that the microarrays described herein may be synthesized by any method known in the art such as those described in U.S. Pat. No. 5,143,854; U.S. Pat. No. 5,424,186; or PCT Publication No. WO 98/20967, all incorporated herein in their entirety by reference. Spot arrays may also be used in which complete probes are “spotted” onto a solid surface as with a computer driven pen plotter, or any other device that transfers small drops of liquid onto specified locations of a solid substrate.

In certain embodiments, linker molecules are attached to a substrate surface on which oligonucleotide sequence arrays are to be synthesized (the linker is an “initiation moiety” a term also broadly including monomers or oligomers on which another monomer can be added). The methods for synthesis of oligonucleotides are known, McBride et al., Tetrahedron Letter 24, 245-248 (1983). Each linker molecule contains a reactive functional group, such as 5′-OH, protected by an acid-labile protecting group. Next, a photo-acid precursor or a photo-acid precursor and its photosensitizer are applied to the substrate. A predetermined light pattern is then projected onto the substrate surface. Acids are produced at the illuminated sites, causing cleavage of the acid-labile protecting group (such as DMT) from the 5′-OH, and the terminal OH groups are free to react with incoming monomers, “monomers” as used hereafter are broadly defined as chemical entities, which, as defined by chemical structures, may be monomers or oligomers or their derivatives. A negligible amount of acid or none is produced at the dark (i.e. non-illuminated) sites and, therefore, the acid labile protecting groups of the linker molecules remain intact. A negligible amount is an amount of acid insufficient to lower the pH to the levels necessary to cause cleavage of a significant number of acid labile protecting groups. The substrate surface is then washed and subsequently contacted with the first monomer (e.g., a nucleophosphoramidite, a nucleophosphonate or an analog compound which is capable of chain growing), which adds only to the deprotected linker molecules under conventional coupling reaction conditions. A chemical bond is thus formed between the OH groups of the linker molecules and an unprotected reactive site (e.g., phosphorus) of the monomers, for example, a phosphite linkage. After proper washing, oxidation and capping steps, the addition of the first residue is complete.

The attached nucleotide monomer also contains a reactive functional terminal group protected by an acid-labile group. The substrate containing the array of growing sequences is then supplied with a second batch of a photo-acid precursor and exposed to a second predetermined light pattern. The selected sequences at irradiated features are deprotected and the substrate is washed and subsequently supplied with the second monomer. Again, the second monomer propagates the nascent oligomer only at the surface sites that have been exposed to light. The second residue added to the growing sequences also contains a reactive functional terminal group protected by an acid-labile group. This chain propagation process is repeated until polymers of desired lengths and desired chemical sequences are formed at all selected surface sites. For a chip containing an oligonucleotide array of any designated sequence pattern, the maximum number of reaction steps is 4×n, where n is the chain length and 4 is a constant for natural nucleotides. Arrays containing modified sequences may require more than 4×n steps.

Photo-acid precursors that may be used as described herein include any compound that produces a photo-generated acid (PGA) upon irradiation. Examples of such compounds include diazoketones, triarylsulfonium, iodonium salts, o-nitrobenzyloxycarbonate compounds, triazine derivatives and the like. Sus et al., Liebigs Ann. Chem. 556: 65-84 (1944); Hisashi Sugiyama et al., U.S. Pat. No. 5,158,885 (1997); Cameron et al., J. Am. Chem. Soc. 113: 4303-4313 (1991); Frechet, Pure & Appl. Chem. 64: 1239-1248 (1992); Patchornik et al., J. Am. Chem. Soc. 92: 6333-6335 (1970). Example of photo-acid precursors include triarylsulfonium hexafluoroantimonate derivatives (Dektar et al. J. Org. Chem. 53: 1835-1837 (1988); Welsh et al., J. Org. Chem. 57: 4179-4184 (1992); DeVoe et al., Advances in Photochemistry 17: 313-355 (1992)). This compound belongs to a family of onium salts, which undergo photodecompositions, either directly or sensitized, to form free radical species and finally produce diarylsulfides and H+. Another example of a photo-acid precursor is diazonaphthoquionesulfonate triester ester, which produces indenecarboxylic acid upon UV irradiation at wavelengths >350 nm. The formation of the acid is due to a Wolff rearrangement through a carbene species to form a ketene intermediate and the subsequent hydration of ketene (Sus et al., Liebigs Ann. Chem. 556: 65-84 (1944); Hisashi Sugiyama et al., U.S. Pat. No. 5,158,885 (1997))

Photo-base precursors may also be used in the practice of the present disclosure. Photo-base precursors include any compound that produces photo-generated base (PGB) upon irradiation. Examples of such compounds include o-benzocarbamates, benzoinylcarbamates, nitrobenzyloxyamine derivatives, and the like. In general, compounds containing amino groups protected by photolabile groups can release amines in quantitative yields. The photoproducts of these reactions, i.e., in situ generated amine compounds, are, in this invention, the basic reagents useful for further reactions.

While the methods of this invention have been described in terms of preferred embodiments, it will be apparent to those of skill in the art that variations may be applied to the methods and in the steps or in the sequence of steps of the methods described herein without departing from the concept, spirit and scope of the invention. All such similar substitutes and modifications apparent to those skilled in the art are deemed to be within the spirit, scope and concept of the invention as defined by the appended claims.

1. An interactive method of providing an array of nucleic acid sequences comprising:
preparing a database of gene probes or probe identifiers wherein each gene probe is effective to identify a specific target gene, and further wherein each of the gene probes is identified by one or more identifiers;
providing an electronic connection to the database effective to allow a user to electronically query the database by the identifiers;
providing means for storing a list of gene probes;
transmitting a collection of selected gene probes from the user to the host; and
producing a nucleic acid probe array that comprises the selected gene probes.

2. The method of claim 1, wherein the identifiers are accession numbers, accession alias numbers, disease names, disease synonym names, gene functions, gene names, gene symbols, gene descriptions, PFAM names, gene ontology (GO) molecular function terms, or locus I.D. numbers.

3. The method of claim 1, wherein the gene probes comprise sets of 1-5 nucleic acid sequences.

4. The method of claim 1, wherein the means for storing a list of gene probes is a storage device on a host server.

5. The method of claim 1, wherein the means for storing a list of gene probes is software that directs storage of the gene probes on the user's computer, the host's computer, or both.

6. The method of claim 1, wherein the probes are nucleic acid probes of from 40 to 50 bases in length.

7. The method of claim 1, wherein the probes are nucleic acid probes of 10-100 bases in length.

8. The method of claim 1, wherein the probes are designed to hybridize to their respective target genes in a region within 1500 bases of the 3′ end of the coding sequence of the target gene.

9. The method of claim 1, wherein the probes are optimized for Tm, C+G content, probe secondary structure, dimerization tendency, or combinations thereof.

10. The method of claim 1, wherein the microarray further comprises control sequences.

11. The method of claim 1, wherein the nucleotide probes of the array are made by synthesis of the nucleic acid probes in situ on a solid substrate comprising isolated sites.

12. The method of claim 1, wherein the nucleotide probes are synthesized by steps that comprise:
(a) addition of protecting groups to the reactive chemical groups within the isolated sites wherein the protecting groups are not photo-labile;
(b) contacting the isolated sites with photo-generated agent precursors;
(c) irradiating a selected subset of the isolated sites effective to generate active agents from the precursors within the selected subset and to deprotect reactive chemical groups within the selected subset;
(d) contacting the isolated sites with a selected nucleic acid monomer comprising a free reactive chemical group and a protected reactive chemical group under conditions in which the free reactive chemical group of the monomer bonds to the deprotected reactive chemical group of the selected subset; and
(e) repeating steps b-d until the selected nucleic acid probes are synthesized.

13. The method of claim 1, wherein the database comprises gene probes or identifiers for human, mouse, rat, Xenopus, bacteria, Drosophila Arabidopsis, and Caenorhabditis genes.

14. A system for providing custom nucleic acid microarrays comprising:
a host computer comprising a data storage media;
a database of gene probes stored on the storage media wherein each gene probe is associated with one or more identifiers;
a user connection configured to allow remote users to connect to the host computer and to access the database;
software means to allow a remote user to query the database to generate a list of gene probes; and
means for remotely selecting listed gene probes;
wherein the selected gene probes are provided on a microarray.

15. The system of claim 14, wherein the computer interface is a modem connection, T1 line, satellite access, or network access.

16. The system of claim 14, further comprising means for storing selected gene probes on the host computer, on the remote user's computer or both.
